(19) United States
(12) Patent Application Publication
(10) Pub. No.: US 2007/0166337 A1
(43) Pub. Date: Jul. 19, 2007

(54) Skincare compositions and methods

(75) Inventors: Klaus Treudler, Rienbek (DE); Henning Mallwitz, (US); Timm Schmidt, (US); Jane Evison, (US)

Correspondence Address: ROTHWELL, FIGG, ERNST & MANBECK, P.C., 1425 K STREET, N.W., SUITE 800, WASHINGTON, DC, 20005, US

(73) Assignee: Boots Healthcare International Limited, Nottingham (GB)

(21) Appl. No.: 10/571,757

(22) PCT Filed: Sep. 10, 2004
(86) PCT No.: PCT/GB04/03879

§ 371 Date: Oct. 2, 2006

(30) Foreign Application Priority Data
Sep. 13, 2003 (GB) 0321541.5
Feb. 19, 2004 (GB) 0403702.4
Aug. 31, 2004 (GB) 0419260.5

(51) Int. Cl. A61K 33/40; A61K 31/60

(52) U.S. Cl. 424/401; 424/616; 514/159

(57) ABSTRACT

There is disclosed a skincare composition suitable for topical application to the skin. The composition comprises from 0.5 to 10% by weight of salicylic acid and from 0.5 to 10% of hydrogen peroxide, but is substantially free of other therapeutic agents selected from the group consisting of anti-microbial agents, anti-bacterial agents, anti-viral agents, anti-fungal agents, anthelmintic agents and anti-inflammatory agents. The composition is useful in the treatment of acne.

This invention relates to skincare compositions, in particular compositions effective in the treatment of acne vulgaris, and to methods of treatment of the skin that involve the application of such compositions.

Acne vulgaris (acne) is a chronic inflammatory condition of the pilosebaceous units of the skin, which is particularly prevalent in adolescents. The condition generally causes the formation, on the skin, of comedones, red papules, pustules and sometimes cysts. This is unsightly and furthermore, if untreated, acne can lead to scarring of the skin. The major causes of acne are thought to be an increase in sebum production, an increased presence of Propionibactedum acne (P. acne), blockage of the pilosebaceus duct and the production of inflammation.

Salicylic acid is known to be effective in the treatment of acne. It is a topical keratolytic agent that works by dissolving the intercellular cement that holds epithelial cells together. Salicylic acid is used in a variety of over-the-counter acne remedies.

It has now been found that an improved topical acne treatment can be achieved by combining salicylic acid with hydrogen peroxide.

Hydrogen peroxide has also been employed in cleansing compositions for topical application to the skin. However, hydrogen peroxide is generally regarded merely as a disinfectant, and has not been employed as an active agent in the treatment of acne.

Topical skincare formulations comprising salicylic acid and hydrogen peroxide have been disclosed, but in such cases the salicylic acid and hydrogen peroxide are merely adjuncts to other therapeutic agents. For example, the following related US patents and patent applications:
U.S. Pat. No. 6,071,541
U.S. Pat. No. 6,296,880
U.S. Pat. No. 6,383,523
US 2002/0172719
   all relate to skin cleansing compositions for a variety of dermatological conditions that comprise hydrogen peroxide and an acidic component (salicylic acid being one example of such an acidic component), in combination with an anti-microbial agent, optionally with an anti-inflammatory agent. The anti-microbial agent includes an antibacterial compound, an antiviral agent, an antifungal agent or an anthelmintic agent. In addition, US 2002/0054918 relates to a topical anti-inflammatory agent comprising hydrogen peroxide in an amount to cleanse the skin, a moisturising agent and an anti-inflammatory agent, optionally with an acidic exfoliant (such as salicylic acid). In the above compositions, the acidic component is said to be present in an amount sufficient to exfoliate, ie remove dead or dying skin cells, from at least a portion of the skin, the hydrogen peroxide is present in an amount sufficient to cleanse at least a portion of the skin and the anti-microbial agent inhibits the formation, and may further reduce the presence of, microbes that cause redness, inflammation and irritation of the skin.

Surprisingly, it has now been found that skincare compositions comprising therapeutically effective concentrations of both salicylic acid and hydrogen peroxide are effective in the treatment of acne without requiring the presence of either an anti-microbial or an anti-inflammatory therapeutic agent. In particular, the combination of salicylic acid and hydrogen peroxide is believed to have valuable therapeutic properties in reducing the presence of P. acne on the skin, especially resulting from the oxidation effect of the hydrogen peroxide.

In view of the above recited art, the provision of an efficacious acne treatment composition containing salicylic acid and hydrogen peroxide without the anti-microbial and /or anti-inflammatory agent is unexpected.

Furthermore, reducing the number of active ingredients in a composition is of significant advantage as it facilitates formulation and also manufacturing processes. The combination of these two ingredients also allows the preparation of a wide variety of non-irritant, stable and cosmetically acceptable therapeutic compositions, including, but not limited to, compositions comprising detergent systems. The combination also provides efficacious compositions for topical application which may be adapted either for leaving on the skin after being applied thereto or for being rinsed off after application.

Thus, according to a first aspect of the invention there is provided a skincare composition suitable for topical application to the skin, the composition comprising from 0.5 to 10% by weight of salicylic acid or a salt thereof, and from 0.5 to 10% by weight of hydrogen peroxide or a compound capable, in use, of generating hydrogen peroxide, wherein the composition is substantially free of other therapeutic agents selected from the group consisting of anti-microbial agents, anti-bacterial agents, anti-viral agents, anti-fungal agents, anthelmintic agents and anti-inflammatory agents.

By “substantially free of other therapeutic agents selected from the group consisting of anti-microbial agents, anti-bacterial agents, anti-viral agents, anti-fungal agents, anthelmintic agents and anti-inflammatory agents” is meant that the composition comprises no effective amount of said compounds. In general, this means that the composition will contain less than 0.01% by weight of any such compound, more preferably less than 0.001% by weight. Preferred compositions are substantially free of any compound (other than salicylic acid and hydrogen peroxide) that is recognised as having a therapeutic effect in the treatment of acne or other dermatological conditions when applied topically to the skin. In further preferred compositions, specific classes of therapeutic agent that are (with the exceptions of salicylic acid and hydrogen peroxide, to the extent that either of such compounds fall within these classes) absent from the composition according to the invention may be:

Antimicrobial or antibacterial compounds, in particular selected from the following:
triclosan, neomycin, clindamycin, polymyxin, bacitracin, benzoyl peroxide, tetracylines such as doxycycline or minocycline, sulfa drugs such as sulfacetamide, penicillins, cephalosporins such as cephalexin, and quinolones such as lomefloxacin, olfoxacin or trovafloxacin.

Antiviral compounds, in particular selected from acyclovir, tamvir, and penciclovir.

Antifungal compounds, in particular selected from the following: famesol, clotrimazole, ketoconazole, econazole, fluconazole, calcium or zinc undecylenate, undecylenic acid, butenafine hydrochloride, ciclopirox olaimine, miconazole nitrate, nystatin, sulconazole, and terbinafine hydrochloride.

Anti-inflammatory compounds, in particular selected from the following: steroidal agents selected from hydrocortisone, fluocinolone acetonide, halcinonide, halobetasol propionate, clobetasol propionate, betamethasone dipropionate, betamethasone valerate, and triamcinolone acetonide, and non-steroidal anti-inflammatory agents selected from aspirin, ibuprofen, ketoprofen, naproxen, aloe vera gel, aloe vera, licorice extract, pilewort, Canadian willow root, zinc, and allantoin.

Anthelmintic compounds, in particular metronidazole.

The compositions according to the invention may be substantially free of all other therapeutic agents. Preferred compositions are entirely free of other therapeutic agents.

In preferred compositions, salicylic acid and hydrogen peroxide are the sole active ingredients having a therapeutic effect in the topical treatment of acne.

In one embodiment of the invention, salicylic acid and hydrogen peroxide are the sole active ingredients in the composition.

Salicylic acid is preferably incorporated into the composition according to the invention as the free acid. However, the pH of the composition may, and generally will, be such that the salicylic acid exists in the composition in dissociated form. As the composition may well contain cationic counterions, the salicylic acid may then be thought of as being present in salt form. Alternatively, the salicylic acid may be incorporated into the composition in salt form, eg as a salt with a Group I metal, such as sodium salicylate. As used herein, unless the context requires otherwise, any and all references to salicylic acid should be taken to encompass references to the acid and to dissociated forms and salts thereof.

The concentration of salicylic acid in the composition according to the invention is preferably at least 1.0% by weight, more preferably at least 1.5% and most preferably at least 1.8% by weight. The concentration of salicylic acid is preferably less than 5% by weight, more preferably less than 4% by weight, and most preferably less than 3% by weight. The concentration of salicylic acid may therefore fall in the range 1.0% to 5% by weight, more preferably 1.5% to 4%, and most preferably 1.8% to 3% by weight. A particularly preferred concentration of salicylic acid is 2% by weight.

The composition most preferably comprises hydrogen peroxide. Alternatively, the composition may comprise a compound that, in use, is capable of generating hydrogen peroxide. An example of the latter class of compound is an adduct such as urea peroxide (carbamide peroxide).

The concentration of hydrogen peroxide in the composition according to the invention is preferably at least 1% by weight. The concentration of hydrogen peroxide is preferably- less than 5% by weight, more preferably less than 3% by weight, and most preferably less than 2% by weight. The concentration of hydrogen peroxide may therefore fall within the range 1% to 5% by weight, more preferably 1% to 3%, and most preferably 1% to 2% by weight:

Preferably, the ratio of salicylic acid to hydrogen peroxide in a composition according to the invention is in the range from 10:1 to 1:10 parts by weight, preferably from 5:1 to 1:2 parts by weight, and most preferably from 2:1 to 1:1 parts by weight.

The composition is preferably prepared with a pH in the range 2.3 to 6.0, more preferably 2.5 to 5.0, and particularly a pH in the range 2.5 to 4.0, eg about pH 3.0 or pH 3.5.

In many instances, it is preferred that the composition should comprise a chelating or sequestering agent, or other agent capable of complexation or other interaction with metal ions present in the composition. Such agents may improve the stability of the composition, and in particular may inhibit or prevent degradation of the hydrogen peroxide. Examples of chelating or sequestering agents include ethylenediamine tetraacetic acid and its salts, notably the dipotassium and especially the disodium salt. Another agent that may perform a similar function is sodium stannate.

The composition according to the invention may be formulated in numerous forms. However, the composition may often take the form of an aqueous or oily solution or dispersion or emulsion or a gel. An emulsion may be an oil-in-water emulsion or a water-in-oil emulsion.

The oil phase of water-in-oil or oil-in-water emulsions may comprise for example:
a) hydrocarbon oils such as paraffin or mineral oils;
b) waxes such as beeswax or paraffin wax;
c) natural oils such as sunflower oil, apricot kernel oil, shea butter or jojoba oil;
d) silicone oils such as dimethicone, cyclomethicone or cetyldimethicone;
e) fatty acid esters such as isopropyl palmitate, isopropyl myristate, dioctylmaleate, glyceryl oleate and cetostearyl isononanoate;
f) fatty alcohols such as cetyl alcohol or stearyl alcohol and mixtures thereof (eg cetearyl alcohol);
g) polypropylene glycol or polyethylene glycol ethers, eg PPG-14 butyl ether; or
h) mixtures thereof, for example, the blend of waxes available commercially under the trade name Cutina (Henkel).

Emulsifiers used may be any emulsifiers known in the art for use in water-in-oil or oil-in-water emulsions. Known cosmetically acceptable emulsifiers include:
a) sesquioleates such as sorbitan sesquioleate, available commercially for example under the trade name Arlacel 83 (ICI), or polyglyceryl-2-sesquioleate;
b) ethoxylated esters of derivatives of natural oils such as the polyethoxylated ester of hydrogenated castor oil available commercially for example under the trade name Arlacel 989 (ICI);
c) silicone emulsifiers such as silicone polyols available commercially for example under the trade name ABIL WS08 (Th. Goldschmidt AG);
d) anionic emulsifiers such as fatty acid soaps e.g. potassium stearate and fatty acid sulphates e.g. sodium cetostearyl sulphate available commercially under the trade name Dehydag (Henkel);
e) ethoxylated fatty alcohols, for example the emulsifiers available commercially under the trade name Brij (ICI);
f) sorbitan esters, for example the emulsifiers available commercially under the trade name Span (ICI);
g) ethoxylated sorbitan esters, for example the emulsifiers available commercially under the trade name Tween (ICI);
h) ethoxylated fatty acid esters such as ethoxylated stearates, for example the emulsifiers available commercially under the trade name Myrj (ICI);
i) ethoxylated mono-, di-, and tri-glycerides, for example the emulsifiers available commercially under the trade name Labrafil (Alfa Chem.);
j) non-ionic sef-emulsifying waxes, for example the wax available commercially under the trade name Polawax (Croda);
k) ethoxylated fatty acids, for example, the emulsifiers available commercially under the trade name Tefose (Alfa Chem.);
l) methylglucose esters such as polyglycerol-3 methyl glucose distearate available commercially under the name Tegocare 450 (Degussa Goldschmidt); or
m) mixtures thereof.

Gels according to the invention may be aqueous or non-aqueous. Aqueous gels are preferred. The gel will contain a thickening agent or gelling agent in order to give sufficient viscosity to the gel. A variety of thickening agents may be used according to the nature of the liquid carrier and the viscosity required and these are recited hereinafter. A particularly suitable thickener is a copolymer of acryloyl dimethyl tauric acid (or a salt thereof), preferably a copolymer of that monomer with another vinylic monomer. For example, the thickening agent is a copolymer of a salt of acryloyl dimethyl tauric acid with another vinylic monomer. The salt may be a salt of a Group I alkali metal, but is more preferably an ammonium salt. Examples of suitable copolymer thickening agents are:
i) Ammonium acryloyl dimethyl taurate I vinyl pyrrolidone copolymer, ie a copolymer of ammonium acryloyl dimethyl taurate and vinyl pyrrolidone (1-vinyl-2-pyrrolidone). This material is available under the trade name Aristoflex AVC from Clariant GmbH, Functional Chemicals Division, D-65840 Sulzbach, Germany.
ii) Ammonium acryloyl dimethyl taurate/Beheneth-25 methacrylate copolymer, ie a copolymer of ammonium acryloyl dimethyl taurate and Beheneth-25 methacrylate, the structure of which is
   CH₂═CH(CH₃)CO₂—(CH₂CH₂O)ₙCH₂(CH₂)₂₀CH₃
   in which n is approximately 25. This material is also available from Clariant GmbH under the trade name Aristoflex HMB.
iii) Ammonium acryloyidimethyltaurate/vinyl formamide copolymer, ie a copolymer of ammonium acryloyl dimethyl taurate and vinyl formamide. Again, a suitable material is available from Clariant GmbH under the trade name Aristoflex AVC-1.

The gel most preferably comprises less than 10% by weight of the thickening agent, and more commonly less than 5% by weight. The amount of thickening agent will generally be greater than 0.1% by weight and more commonly greater than 0.5% by weight. The amount of thickening agent in the gel will preferably lie in the range 0.1 to 5% by weight, more preferably 0.5 to 5% by weight. Typically, the amount of thickening agent will be less than 3% by weight, eg about 1% by weight or about 2% by weight.

The gel according to the invention preferably has a viscosity of from about 50 mPa·s to about 20,000 mPa·s, more preferably from about 100 mPa·s to about 10,000 mPa·s. Viscosity may be measured using a Brookfield RVT viscometer equipped with a spindle 4 rotating at 10 rpm after 2 minutes.

In the case of solutions or dispersions, and gels, the composition will generally contain a solvent system or other continuous liquid phase. Such a system is preferably aqueous. However, mixed solvent systems may often be used with advantage. Such a mixed solvent system most preferably comprises water, in admixture with a co-solvent, most preferably a lower (eg C₁₋₆) alcohol, in particular ethanol.

Preferred aqueous systems comprise water in an amount of at least 50% by weight, more preferably at least 60% by weight, most preferably at least 70% by weight and especially at least 80% by weight. The upper limit of water will depend on the amounts of other ingredients incorporated in the composition so that the water may form the remainder of the composition up to 100% of the composition. A typical maximum value is less than 90% by weight, for example 80% by weight or 85% by weight.

The composition most preferably comprises in excess of 5% by weight of the cosolvent, and may comprise in excess of 10% by weight, in excess of 20% by weight, or in excess of 30% by weight of the cosolvent. The amount of cosolvent present in the composition preferably does not exceed 50% by weight. The amount of cosolvent thus preferably lies in the range 5% to 50% by weight, more preferably 10% to 50% by weight. In general, higher proportions of cosolvent may be required in compositions containing higher proportions of ingredients (eg topically active ingredients, as discussed below) that are of low solubility in water. Where such ingredients are absent, or their concentration is relatively low, the proportion of cosolvent may also be somewhat lower than in other embodiments, eg up to 20% by weight.

The composition may additionally comprise other skincare active agents which are well known in the art which may be effective to aid the normal functioning of the skin. One group of preferred compositions comprise hydrolysed milk protein to regulate sebum production.

The composition may additionally comprise other components which will be well known to those skilled in the art. These include, for example:
a) Emollients—ingredients that help to maintain the soft, smooth and pliable appearance of skin. Such ingredients may function by their ability to remain on the surface of the skin or in the stratum corneum, and to act as lubricants, reducing or preventing flaking of the skin and improving the skin's appearance. Examples of emollients are isopropyl myristate, triglycerides of fatty acids eg lauric triglyceride or capric/caprylic triglyceride, such as the triglyceride available commercially under the trade name Miglyol 810 (Huls UK), and the polypropylene glycol ether of stearyl alcohol known as PPF-15 Stearyl Ether.

Particularly preferred emollients are polysiloxane compounds, in particular those known as cyclomethicone, ie cyclic dimethyl polysiloxane compounds that conform to the formula:
—(Si(CH₃)₂)n-
in which n has a value between 3 and 7.
b) Humectants or Moisturisers—ingredients intended to increase the water content of the top layers of the skin. Examples of such ingredients are glycerin, 1,3-butylene glycol and propylene glycol.
c) Emulsion stabilising salts such as sodium chloride, sodium citrate or magnesium sulphate.
d) Preservatives—ingredients which prevent or retard microbial growth and thus protect the composition from spoilage. Examples of preservatives include such as propylparaben, bronopol, sodium dehydroacetate, polyhexamethylenebiguanide hydrochloride, isothiazolone and diazolidinylurea.
e) Chelating agents or sequestering agents (sequestrants)—ingredients that have the ability to complex with and inactivate metallic ions in order to prevent their adverse effects on the stability or appearance of the composition, as described above. Examples of chelating agents are ethylenediamine tetraacetic acid and its salts, notably the dipotassium and especially the disodium or tetrasodium salt.
f) Abrasives—ingredients used to assist in the removal of unwanted tissue or foreign materials from the skin during application of the composition. Abrasives commonly comprise fine solid particles. One example of a suitable abrasive is polyethylene beads.
g) pH adjusters—Ingredients used to control the pH of the composition. Examples of pH adjusters are inorganic salts such as sodium hydroxide, and organic bases such as triethanolamine.
h) Surfactants—In addition to their use as emulsifying agents, surfactants may be used in compositions according to the invention as cleansing agents, foam boosters or solubilising agents. Many of the emulsifying agents referred to above may be used for these purposes, and other suitable surfactants will be readily apparent to those skilled in the art.
i) Thickeners—ingredients intended to increase the viscosity of the composition. Thickeners that are water-soluble or hydrophilic are preferred, and examples include acrylic acid polymers, eg those available commercially under the trade name Carbopol (B.F. Goodrich), modified celluloses, eg hydroxypropylmethylcellulose or hydroxyethylcellulose available commercially under the trade name Natrosol (Hercules), alkylgalactomanans available under the trade name N-Hance, xanthan gum, cetyl alcohol and sodium chloride.
j) Perfumes and colourings.

The compositions according to the invention may be prepared by standard methods known in the art, for example combining all the ingredients in a single aqueous or non-aqueous phase. The single aqueous phase may include hydroalcoholic systems, optionally with a thickener. In the case of a two phase composition (for example oil and water), all of or a proportion of the oil-soluble ingredients may be combined to form an oily phase and all of or a proportion of the water-soluble ingredients may be combined in an aqueous phase. This may be followed by mixing the oily and aqueous phases, together with any remaining ingredients, to form an emulsion.

The composition according to the invention may be applied and left on the skin to have the desired therapeutic effect or it may be applied and then rinsed off, for example with water. The composition may be applied with the aid of a fibrous material, for example a pad or a wipe.

According to another aspect of the invention, there is provided an article comprising a fibrous substrate, for example a material in the form of a pad or a wipe, impregnated with a skincare composition according to the invention comprising salicylic acid or a salt thereof and hydrolysed milk protein. The fibrous substrate may be used to apply the composition onto the skin.

Preferably, said fibrous substrate is impregnated with the skincare composition in an amount in the range from 10 to 30% by weight, preferably from 15 to 25% by weight and most preferably from 18 to 22% by weight of the fibrous substrate. Suitable fibrous substrates comprise materials which include natural or synthetic fibres or a mixture thereof, for example cellulose and/or cotton fibres. The fibrous substrate may be impregnated with the composition as a wet wipe which is arranged for immediate use to apply the skincare composition of the present invention to the skin of the user. Alternatively, the fibrous substrate may be impregnated with the skincare composition and dried to form a dry wipe which requires to be wetted, for example with water, before it can be used.

According to another aspect of the invention, there is provided a method for the prophylactic or remedial treatment of acne, which method comprises the topical application to the skin of a patient of a skincare composition comprising from 0.5 to 10% by weight of salicylic acid or a salt thereof, and from 0.5 to 10% of hydrogen peroxide or a compound capable, in use, of generating hydrogen peroxide, wherein the composition is substantially free of other therapeutic agents selected from the group consisting of anti-microbial agents, anti-bacterial agents, anti-viral agents, anti-fungal agents, anthelmintic agents and anti-inflammatory agents. In a preferred method, the composition is substantially free of other therapeutic agents.

It will be appreciated that the method according to this aspect of the invention may be a therapeutic method, but will often be a primarily cosmetic method, the objective of which is to reduce or eliminate externally visible, and often unsightly, symptoms of acne vulgaris.

In a further aspect of the invention, there is provided the use of salicylic acid and hydrogen peroxide peroxide substantially free of other therapeutic agents selected from the group consisting of anti-microbial agents, anti-bacterial agents, anti-viral agents, anti-fungal agents, anthelmintic agents and anti-inflammatory agents for the prophylactic or remedial treatment of acne.

In a yet further aspect of the invention, there is provided the use of salicylic acid and hydrogen peroxide as the sole active ingredients in the manufacture of a composition for the prophylactic or remedial treatment of acne by topical application of the composition to the skin.

The invention will now be described in greater detail, by way of illustration only, with reference to the following Examples.

### EXAMPLE 1

Cleansing Anti-Acne Cream

| Ingredients | Trade Name | % w/w |
|---|---|---|
| PPG-14 butyl ether | Ucon Fluid AP | 8.00 |
| Cetearyl alcohol (80%) | Polawax GP 200 | 7.5 |
| and PEG-20 stearate (20%) | | |
| Salicylic acid | | 2.00 |
| Hydrogen peroxide (35% solution) | | 4.286 |
| Parfum | | 0.10 |
| Sodium hydroxide (30% solution) | | 0.050 |
| Sodium stannate | | 0.005 |
| Aqua | | to 100% |

Method

The salicylic acid was dissolved in the PPG-14 butyl ether at a temperature of 70° C. to 75° C. The Polawax GP 200 was then added to this mixture to form the oil phase. The oil phase was then emulsified with the water and sodium stannate at a temperature of 70° C. to 75° C. The resultant emulsion was then cooled to room temperature and then the hydrogen peroxide and parfum were then stirred in separately. Sodium Hydroxide was then added to adjust the pH to 3.

### EXAMPLE 2

Gel

| Ingredients | % w/w |
|---|---|
| Alcohol denat. | 20 |
| Propylene Glycol | 18 |
| Butylene Glycol | 15 |
| Hydroxypropyl Methylcellulose | 2.5 |
| Salicylic Acid | 2.0 |
| Hydrogen peroxide | 1.5 |
| Sodium Citrate | 0.3 |
| Aqua | to 100% |

Method

The hydroxypropyl methylcellulose was homogenised into the water to form a thickened dispersion. A mixture of propylene glycol, butylene glycol, salicylic acid predispersed in the alcohol denat was gently stirred into the aqueous phase, together with the sodium citrate and hydrogen peroxide until a clear gel was formed.

### EXAMPLE 3

Cream Cleaner (Scrub)

| Ingredients | % w/w |
|---|---|
| Cetyl Betaine (30%) | 6.667 |
| PPG-15 Stearyl Ether | 4 |
| Sodium Lauryl Sulfate (28%) | 3.571 |
| Glycerin | 3 |
| Stearyl Alcohol | 2.88 |
| Salicylic Acid | 2.00 |
| Distearyldimonium Chloride | 1.5 |
| Hydrogen peroxide | 1.5 |
| Oxidized Polyethylene | 1.0 |
| Cetyl Alcohol | 0.8 |
| Steareth-21 | 0.5 |
| Behenyl Alcohol | 0.32 |
| PPG-30 | 0.25 |
| Steareth-2 | 0.25 |
| Parfum | 0.2 |
| Menthol | 0.075 |
| Disodium EDTA | 0.01 |
| Aqua | to 100% |

Method

All the ingredients, apart from menthol, parfum, oxidised polyethylene, sodium lauryl sulphate, cetyl betaine, were blended together at 70 to 75° C. to form a uniform mixture. The mixture was cooled to room temperature and then the remaining ingredients were mixed in separately.

### EXAMPLE 4

Lotion

| Ingredient | % w/w |
|---|---|
| Alcohol, Denat. | 37 |
| Isoceteth-20 | 2.86 |
| Salicylic Acid | 2 |
| Hydrogen peroxide | 1.5 |
| Aloe Barbadensis Gel | 0.495 |
| Parfum | 0.3 |
| Triethanolamine | 0.18 |
| Disodium EDTA | 0.005 |
| Imidazolidinyl Urea | 0.004 |
| Methylparaben | 0.00085 |
| Denatonium Benzoate | 0.00023 |
| Propylparaben | 0.00015 |
| Aqua | to 100% |

Method

All the ingredients were mixed at room temperature to form a uniform composition.

The above lotion may be impregnated into mixed natural and synthetic fibre pads in an amount of 95-110 ml per 65 pads (5 cm diameter). The above lotion may also be used in a roller-ball dispenser.

### EXAMPLE 5

Cleansing Wash

| Ingredient | % w/w |
|---|---|
| Sodium Laureth Sulfate | 11.9 |
| Propylene Glycol | 3 |
| Salicylic Acid | 2 |
| Coco Glucoside | 1.5 |
| Hydrogen peroxide | 1.5 |
| Glyceryl Oleate | 1.5 |
| Cocamidopropyl Betaine | 1.4 |
| Parfum | 0.5 |
| Sodium Chloride | 0.27 |
| Polyquaternium-10 | 0.2 |
| Aqua | to 100% |

Method

The salicylic acid was dispersed within the propylene glycol, cocoglucoside and Glyceryl oleate to form a lump free dispersion. The dispersion was then mixed into sodium laureth sulfate which had already been premixed with the water. The rest of the ingredients were then mixed in separately to form the composition.

### EXAMPLE 6

Lotion for Impregnated Wipes

| Ingredient | % w/w |
|---|---|
| PPG-14 butyl ether | 8.0 |
| Glycerin | 5.45 |
| Cetearyl isononanoate | 2.25 |
| Salicylic acid | 2.0 |
| Hydrogen peroxide | 1.5 |
| Ceteareth-20 | 1.125 |
| Cetearyl alcohol | 1.125 |
| Glyceryl Stearate | 0.45 |
| Parfum | 0.2 |
| Cetyl palmitate | 0.15 |
| Ceteareth-12 | 0.15 |
| Disodium EDTA | 0.10 |
| Aloe barbadensis juice | 0.025 |
| Maltodextrin | 0.025 |
| Sodium hydroxide | 0.00012 |
| Aqua | to 100% |

Method

All ingredients, apart from Aloe barbadensis juice, hydrogen peroxide, and parfum, were mixed and heated to 90° C. The mixture was cooled to room temperature with stirring. The remaining ingredients were stirred into the mixture to form a uniform composition.

### EXAMPLE 7

Scrub Wipe

| Ingredients | % w/w |
|---|---|
| PPG-14 butyl ether | 8.00 |
| Cetearyl isononoate | 2.25 |
| Salicylic acid | 2.00 |
| Ceteareth-20 | 1.13 |
| Cetearyl alcohol | 1.13 |
| Glyceryl Stearate | 0.45 |
| Glycerin | 0.45 |
| Hydrolysed Milk Protein | 0.20 |
| Hydrogen peroxide | 1.50 |
| Menthol | 0.10 |
| Disodium EDTA | 0.10 |
| Cetyl palmitate | 0.15 |
| Ceteareth palmitate | 0.15 |
| Parfum | 0.10 |
| Aqua | to 100% |

Method

All ingredients, apart from hydrolysed milk protein, menthol, hydrogen peroxide and parfum, were mixed and heated to 90° C. The mixture was cooled to room temperature with stirring. The remaining ingredients were stirred into the mixture to form a uniform composition.

### EXAMPLE 8

Lotion for Impregnated Pads

| Ingredients | % w/w |
|---|---|
| Ethanol | 37.00 |
| Isoceteth-20 | 3.00 |
| Salicylic acid | 2.00 |
| Hydrogen Peroxide (active 35%) | 4.29 |
| Hydrolysed Milk Protein | 0.20 |
| (mixed with propylene glycol and water) | |
| Sodium hydroxide (30%) | 0.20 |
| Parfum | 0.10 |
| Disodium EDTA | 0.005 |
| Aqua | to 100% |

Method

All the ingredients were mixed at room temperature to form a uniform composition.

### EXAMPLE 9

Gel Lotion

| Ingredients | % w/w |
|---|---|
| Ethanol | 11.5 |
| Glycerin | 0.50 |
| Isoceteth-20 | 1.00 |
| Salicylic acid | 0.50 |
| Hydrogen peroxide (35%) | 4.29 |
| Ammonium acryloyldimethyltaurate/ | 1.50 |
| vinyl pyrrolidone copolymer | |
| Hydrolyzed Milk Protein | 0.20 |
| Sodium hydroxide (30%) | 0.40 |
| Parfum | 0.20 |
| Disodium EDTA | 0.005 |
| Aqua | to 100% |

Method

The salicylic acid was dissolved in the alcohol. Water, glycerin and disodium EDTA were added with mixing. The ammonium acryloyldimethyltaurate/vinyl pyrrolidone copolymer was then added with continuous homogenisation. Finally, the isoceteth-20, hydrogen peroxide, hydrolysed milk peptide and parfum were added in the water. The pH was adjusted to 3 with the sodium hydroxide.

### EXAMPLE 10

Lotion for Impregnated Pads

| Ingredients | % w/w |
|---|---|
| Sorbitol (70%) | 0.50 |
| Denatured ethanol | 37.00 |
| Hydrogen Peroxide | 4.29 |
| Isoceteth-20 | 3.00 |
| Salicylic acid | 2.00 |
| Hydrolyzed Milk Protein | 0.20 |
| Sodium hydroxide (30%) | 0.20 |
| Parfum | 0.10 |
| Disodium EDTA | 0.005 |
| Aqua | to 100% |

Method

All the ingredients were mixed at room temperature to form a uniform composition.

1. A skincare composition suitable for topical application to the skin, the composition comprising from 0.5 to 10% by weight of salicylic acid or a salt thereof, and from 0.5 to 10% by weight of hydrogen peroxide or a compound capable, in use, of generating hydrogen peroxide, wherein the composition is substantially free of other therapeutic agents selected from the group consisting of anti-microbial agents, anti-bacterial agents, anti-viral agents, anti-fungal agents, anthelmintic agents and anti-inflammatory agents.

2. A composition as claimed in claim 1, which comprises salicylic acid.

3. A composition as claimed in claim 2, wherein the concentration of salicylic acid in the composition is at least 1.0% by weight, more preferably at least 1.5% by weight and most preferably at least 1.8% by weight.

4. A composition as claimed in claim 2, wherein the concentration of salicylic acid is less than 5% by weight, more preferably less than 4% by weight, and most preferably less than 3% by weight.

5. A composition as claimed in claim 2, wherein the concentration of salicylic acid is in the range from 1.0% to 5% by weight, more preferably from 1.5% to 4% by weight, and most preferably from 1.8% to 3% by weight.

6. A composition as claimed in claim 1, which comprises hydrogen peroxide.

7. A composition as claimed in claim 6, wherein the concentration of hydrogen peroxide in the composition is at least 1% by weight.

8. A composition as claimed in claim 6, wherein the concentration of hydrogen peroxide is less than 5% by weight, more preferably less than 3% by weight, and most preferably less than 2% by weight.

9. A composition as claimed in claim 6, wherein the concentration of hydrogen peroxide is within the range 1% to 5% by weight, more preferably 1% to 3% by weight, and most preferably 1% to 2% by weight.

10. A composition as claimed in claim 6 wherein the ratio of salicylic acid to hydrogen peroxide is in the range from 10:1 to 1:10 parts by weight, preferably from 5:1 to 1:2 parts by weight, and most preferably from 2:1 to 1:1 parts by weight.

11. A composition as claimed in claim 1, wherein the pH of the composition is in the range 2.3 to 6.0, more preferably 2.5 to 5.0.

12. A composition as claimed in claim 11, wherein the pH is in the range 2.5 to 4.0.

13. A composition as claimed in claim 1, which further comprises a chelating or sequestering agent, or other agent capable of complexation or other interaction with metal ions present in the composition.

14. A composition as claimed in claim 13, wherein the chelating or sequestering agent is ethylenediamine tetraacetic acid or a salt thereof.

15. A composition as claimed in claim 13, wherein said agent is sodium stannate.

16. A composition as claimed in claim 1, which further comprises a thickening agent.

17. A composition as claimed in claim 1, wherein the thickening agent is a copolymer of acryloyl dimethyl tauric acid or a salt thereof.

18. A composition as claimed in claim 1, which has the form of an aqueous or oily solution or dispersion or emulsion or a gel.

19. A composition as claimed in claim 1 which is in the form of an emulsion.

20. A composition as claimed in claim 19, wherein the emulsion is an oil-in-water emulsion.

21. A composition as claimed in claim 19, wherein the emulsion is a water-in-oil emulsion.

22. A composition as claimed in claim 1, which comprises an aqueous solvent system.

23. A composition as claimed in claim 22, wherein the solvent system is a mixed solvent system comprising water in admixture with a co-solvent.

24. A composition as claimed in claim 23, wherein the co-solvent is a lower alcohol, most preferably ethanol.

25. A composition as claimed in claim 1, which comprises one or more excipients selected from the group consisting of emulsifiers, emollients, humectants or moisturisers, binders, conditioning agents, emulsion stabilising salts, preservatives, abrasives, pH adjusters, surfactants, perfumes and colourings.

26. A composition as claimed in claim 1, wherein the composition is substantially free of other therapeutic agents.

27. An article comprising a fibrous substrate impregnated with a skincare composition as claimed in claim 1.

28. An article as claimed in claim 27, wherein the fibrous substrate is impregnated with the skincare composition in an amount in the range from 10 to 30% by weight, preferably from 15 to 25% by weight and most preferably from 18 to 22% by weight of the fibrous substrate.

29. An article as claimed in claim 27 wherein the substrate comprises cellulose or cotton fibres or a mixture thereof.

30. A method for the prophylactic or remedial treatment of acne, which method comprises the topical application to the skin of a patient of a skincare composition comprising from 0.5 to 10% by weight of salicylic acid or a salt thereof, and from 0.5 to 10% of hydrogen peroxide or a compound capable, in use, of generating hydrogen peroxide, wherein the composition is substantially free of other therapeutic agents selected from the group consisting of anti-microbial agents, anti-bacterial agents, anti-viral agents, anti-fungal agents, anthelmintic agents and anti-inflammatory agents.

31. A method as claimed in claim 30, which is a cosmetic method.

32. A method as claimed in claim 30, which is a therapeutic method.

33. The use of salicylic acid and hydrogen peroxide substantially free of other therapeutic agents selected from the group consisting of anti-microbial agents, anti-bacterial agents, anti-viral agents, anti-fungal agents, anthelmintic agents and anti-inflammatory agents for the prophylactic or remedial treatment of acne.

34. The use of salicylic acid and hydrogen peroxide as the sole active ingredients in the manufacture of a composition for the prophylactic or remedial treatment of acne by topical application of the composition to the skin.
